(19) United States
(12) Patent Application Publication
(10) Pub. No.: US 2024/0347201 A1
(43) Pub. Date: Oct. 17, 2024

(54) INFORMATION PROCESSING DEVICE, TABLET TERMINAL, OPERATING METHOD FOR INFORMATION PROCESSING DEVICE, INFORMATION PROCESSING PROGRAM, AND RECORDING MEDIUM

(71) Applicant: FUJIFILM Corporation, Tokyo (JP)

(72) Inventor: Kenichi HARADA, Kanagawa (JP)

(73) Assignee: FUJIFILM Corporation, Tokyo (JP)

(21) Appl. No.: 18/747,433

(22) Filed: Jun. 18, 2024

Related U.S. Application Data
(63) Continuation of application No. PCT/JP2022/040671, filed on Oct. 31, 2022.

(30) Foreign Application Priority Data
Dec. 27, 2021 (JP) 2021-212815

(51) Int. Cl. G16H 50/20; G06F 40/242; G06T 7/00; G10L 15/22

(52) U.S. Cl. CPC G16H 50/20; G06F 40/242; G06T 7/0012; G10L 15/22; G06T 2207/10068; G06T 2207/30096; G10L 2015/223

(57) ABSTRACT

Provided are an information processing device, a tablet terminal, an operating method for an information processing device, an information processing program, and a recording medium with which record information related to endoscopy can be acquired in a stress-free manner using natural utterances during endoscopy. The tablet terminal includes a processor (110) and a first dictionary (122) in which record information to be recorded in relation to endoscopy is registered. The first dictionary (122) is configured such that identifying characters that differ from the record information and the record information are associated with each other. The processor (110) recognizes speech which is uttered by a user during endoscopy and which expresses the identifying characters, and acquires the record information corresponding to the identifying characters from the first dictionary (122) on the basis of the recognized identifying characters.

### CROSS-REFERENCE TO RELATED APPLICATIONS

The present application is a Continuation of PCT International Application No. PCT/JP2022/040671 filed on Oct. 31, 2022 claiming priority under 35 U.S.C § 119 (a) to Japanese Patent Application No. 2021-212815 filed on Dec. 27, 2021. Each of the above applications is hereby expressly incorporated by reference, in its entirety, into the present application.

### BACKGROUND OF THE INVENTION

### 1. Field of the Invention

The present invention relates to an information processing device, a tablet terminal, an operating method for an information processing device, an information processing program, and a recording medium, and more particularly, to a technology for inputting, through voice operation, record information to be recorded in relation to endoscopy.

### 2. Description of the Related Art

During endoscopy, a physician is in a state of operating an endoscope with both hands and using foot switches with both feet. If the physician wants to operate additional equipment, voice operation would be an effective means of doing so.

Heretofore, in the technical field of performing examination and diagnostic support using medical images, it has been known to recognize speech uttered by a user and perform processing based on a recognition result. For example, JP1996-052105A (JP-H08-052105A) describes operating an endoscope by voice input. Also, JP2004-102509A describes providing voice input for report creation.

### SUMMARY OF THE INVENTION

However, in some cases, the patient is not anesthetized or given painkillers during endoscopy, and therefore words that the patient would be afraid to hear (especially the names of diagnoses of serious illnesses) are difficult to adopt as words for voice operation. There is also a problem in that while record information, such as the names of diagnoses, procedures, and treatment tools to be recorded in a diagnostic report, is recorded by formal names, some names are also long, and therefore voice input of record information using formal names is not user-friendly.

The present invention has been devised in the light of such circumstances, and an objective thereof is to provide an information processing device, a tablet terminal, an operating method for an information processing device, an information processing program, and a recording medium with which record information related to endoscopy can be acquired in a stress-free manner using natural utterances during endoscopy.

To achieve the above objective, the invention as in a first aspect is an information processing device including a processor and a first dictionary in which record information to be recorded in relation to endoscopy is registered. The first dictionary is configured such that identifying characters that differ from the record information and the record information are associated directly or indirectly, and the processor recognizes speech which is uttered by a user during endoscopy and which expresses the identifying characters, and acquires the record information corresponding to the identifying characters from the first dictionary on the basis of the recognized identifying characters.

According to the first aspect of the present invention, when acquiring record information related to endoscopy by voice operation during endoscopy, the user (physician) does not utter the record information, but instead utters identifying characters associated with the record information. The processor recognizes speech expressing the identifying characters uttered by the user, and acquires record information corresponding to the identifying characters from the first dictionary on the basis of the identifying characters obtained by speech recognition. This allows for the acquisition of record information without requiring the user to utter words that the patient would be afraid to hear (such as the names of diagnoses of serious illnesses, for example), and the acquisition of record information with the record information being in formal names even if the user utters abbreviations, words, and the like that the user is normally accustomed to using.

In an information processing device according to a second aspect of the present invention, preferably, the processor acquires an endoscopic image related to the record information during the endoscopy, and saves the acquired endoscopic image and the record information in association with each other in a memory.

In an information processing device according to a third aspect of the present invention, preferably, the first dictionary includes at least one of a diagnosis name dictionary containing names of diagnoses indicating lesions as the record information, a treatment name dictionary containing names of treatments indicating treatments involving an endoscope as the record information, or a treatment tool name dictionary containing names of treatment tools indicating endoscope treatment tools as the record information.

In an information processing device according to a fourth aspect of the present invention, preferably, the identifying characters include at least one of numerals, single letters of the alphabet, or abbreviations or common names indicating the record information.

In an information processing device according to a fifth aspect of the present invention, preferably, the first dictionary is formed from a second dictionary in which identification information indicating the record information and the record information are registered in association with each other and a third dictionary in which the identifying characters and the identification information are registered in association with each other, and the processor acquires the identification information associated with the identifying characters from the third dictionary on the basis of the recognized identifying characters, and acquires the record information associated with the identification information from the second dictionary on the basis of the acquired identification information. The third dictionary can be custom user dictionaries (multiple dictionaries) for multiple users. In this case, the second dictionary can be used in common among the multiple users.

In an information processing device according to a sixth aspect of the present invention, preferably, a graphical user interface (GUI) is further included, and the processor newly creates the third dictionary or edits registered content of the third dictionary by operation input from the GUI.

In an information processing device according to a seventh aspect of the present invention, preferably, a graphical user interface (GUI) is further included, and the processor sets the first dictionary to enabled or disabled by operation input from the GUI.

In an information processing device according to an eighth aspect of the present invention, preferably, the processor acquires an endoscopic image during the endoscopy, and enables the first dictionary when a specific type of photographic subject is detected from the endoscopic image. For example, in the case where a specific type of photographic subject (for example, a neoplastic lesion) is to be detected, the first dictionary can be enabled so that record information cannot be acquired through the utterance of words that the patient would be afraid to hear (the names of diagnoses related to neoplasms).

In an information processing device according to a ninth aspect of the present invention, preferably, the processor acquires an endoscopic image during the endoscopy, detects a type of lesion from the endoscopic image, and sets the first dictionary to enabled or disabled according to the detected type of lesion. This allows for more fine-grained settings for enabling or disabling the first dictionary.

In an information processing device according to a tenth aspect of the present invention, preferably, a communication unit that communicates with a server that provides a speech recognition engine is further included. The processor downloads or updates the speech recognition engine from the server through the communication unit, and recognizes speech uttered by the user by using the downloaded or updated speech recognition engine. This eliminates the need to prepare a speech recognition engine in advance on the information processing device side, and also allows for the acquisition of the latest speech recognition engine. This also allows for the acquisition of a speech recognition device suited to the attributes of the user.

In an information processing device according to an eleventh aspect of the present invention, preferably, the first dictionary includes a diagnosis name dictionary containing a plurality of names of diagnoses indicating lesions and a treatment tool name dictionary containing a plurality of names of treatment tools indicating endoscope treatment tools, and the processor acquires an endoscopic image during the endoscopy, recognizes at least one of a lesion or a treatment tool used in a treatment involving an endoscope on the basis of the endoscopic image, selects the diagnosis name dictionary or the treatment tool name dictionary on the basis of a result of recognizing the lesion or the treatment tool, and acquires the record information corresponding to the identifying characters from the selected dictionary on the basis of the recognized identifying characters. By automatically selecting the dictionary to be used, candidates of the identifying characters to be obtained by speech recognition can be narrowed down, and misrecognition in speech recognition can be reduced.

In an information processing device according to a twelfth aspect of the present invention, preferably, the processor, upon recognizing speech expressing a wake word during the endoscopy, recognizes speech expressing the identifying characters uttered thereafter. This can keep unintended user speech from being recognized.

In an information processing device according to a thirteenth aspect of the present invention, preferably, the first dictionary includes at least one of a diagnosis name dictionary containing a plurality of names of diagnoses indicating lesions, a treatment name dictionary containing a plurality of names of treatments indicating treatments involving an endoscope, or a treatment tool name dictionary containing a plurality of names of treatment tools indicating endoscope treatment tools, the wake word is a word specifying at least one dictionary from among the diagnosis name dictionary, the treatment name dictionary, and the treatment tool name dictionary, and the processor acquires the record information corresponding to the identifying characters from the dictionary specified by the wake word, on the basis of the recognized identifying characters. This can keep unintended user speech from being recognized, and since a dictionary is specified at the same, candidates of the identifying characters to be obtained by speech recognition can be narrowed down, thereby suppressing misrecognition in speech recognition.

In an information processing device according to a fourteenth aspect of the present invention, preferably, a second display device independent from a first display device on which an endoscopic image is displayed during the endoscopy is further included, and the processor displays the first dictionary on the second display device during the endoscopy. This allows the user to confirm the identifying characters associated with desired record information while the user is looking at the first dictionary, and utter speech expressing the confirmed identifying characters.

In an information processing device according to a fifteenth aspect of the present invention, preferably, the processor displays on the second display device at least one of a result of recognizing the speech uttered by the user or the acquired record information.

In an information processing device according to a sixteenth aspect of the present invention, preferably, a masking sound generating device that generates masking sound that inhibits the ability of a patient to hear the speech uttered by the user during the endoscopy is further included.

The invention as in a seventeenth aspect is a tablet terminal including the information processing device according to any of the first to fifteenth aspects of the present invention.

The invention as in an eighteenth aspect is an operating method for an information processing device including a processor and a first dictionary in which record information to be recorded in relation to endoscopy is registered, the first dictionary being configured such that identifying characters that differ from the record information and the record information are associated directly or indirectly. The operating method includes: recognizing, by the processor, speech which is uttered by a user during endoscopy and which expresses the identifying characters; and acquiring, by the processor, the record information corresponding to the identifying characters from the first dictionary on the basis of the recognized identifying characters.

The invention as in a nineteenth aspect is an information processing program causing a computer to execute the operating method for an information processing device according to the eighteenth aspect.

The invention as in a twentieth aspect is a non-transitory and computer-readable recording medium in which the information processing program according to the nineteenth aspect of the present invention is recorded.

According to the present invention, record information related to endoscopy can be acquired in a stress-free manner using natural utterances during endoscopy.

### BRIEF DESCRIPTION OF THE DRAWINGS

FIG. 1 is a system configuration diagram including a tablet terminal that functions as an information processing device and an endoscope system according to the present invention;

FIG. 2 is a block diagram illustrating an embodiment of a hardware configuration of a processor device that forms the endoscope system illustrated in FIG. 1;

FIG. 3 is a diagram illustrating an example of a display screen on a first display device that forms the endoscope system illustrated in FIG. 1;

FIG. 4 is a block diagram illustrating an embodiment of a hardware configuration of the tablet terminal illustrated in FIG. 1;

FIG. 5 is a functional block diagram illustrating a first embodiment of a tablet terminal;

FIG. 6 is a diagram illustrating an example of a diagnosis name dictionary which is a first dictionary saved in a memory of a tablet terminal;

FIG. 7 is a diagram illustrating an example of a treatment name dictionary which is a first dictionary saved in a memory of a tablet terminal;

FIG. 8 is a diagram illustrating an example of a treatment tool name dictionary which is a first dictionary saved in a memory of a tablet terminal;

FIG. 9 is a functional block diagram illustrating a second embodiment of a tablet terminal;

FIG. 10 is a diagram illustrating an example of a diagnosis name dictionary which is a second dictionary saved in a memory of a tablet terminal;

FIG. 11 is a diagram illustrating an example of a treatment name dictionary which is a second dictionary saved in a memory of a tablet terminal;

FIG. 12 is a diagram illustrating an example of a treatment tool name dictionary which is a second dictionary saved in a memory of a tablet terminal;

FIG. 13 is a diagram illustrating an example of a third dictionary saved in a memory of a tablet terminal;

FIG. 14 is a flowchart illustrating a procedure for using a tablet terminal to create a third dictionary;

FIG. 15 is a flowchart illustrating the flow of setting a first dictionary to enabled/disabled and acquiring record information in a tablet terminal;

FIG. 16 is a flowchart illustrating an example of automatically setting a first dictionary to enabled/disabled in a tablet terminal;

FIG. 17 is a flowchart illustrating another example of automatically setting a first dictionary to enabled/disabled in a tablet terminal;

FIG. 18 is a flowchart illustrating a procedure by which a tablet terminal acquires a speech recognition engine;

FIG. 19 is a flowchart illustrating an example of utilizing speech recognition of a wake word;

FIG. 20 is a flowchart illustrating another example of utilizing speech recognition of a wake word;

FIG. 21 is a flowchart illustrating an example of automatically selecting a diagnosis name dictionary and a treatment tool name dictionary;

FIG. 22 is a diagram illustrating an example of a display screen on a tablet terminal during endoscopy;

FIG. 23 is a diagram illustrating an example of a first dictionary displayed on the display screen in FIG. 22; and

FIG. 24 is a diagram illustrating an example of an examination room in which a masking sound generating device is disposed.

### DESCRIPTION OF THE PREFERRED EMBODIMENTS

The following describes preferred embodiments of an information processing device, a tablet terminal, an operating method for an information processing device, an information processing program, and a recording medium according to the present invention, in accordance with the attached drawings.

### System Configuration

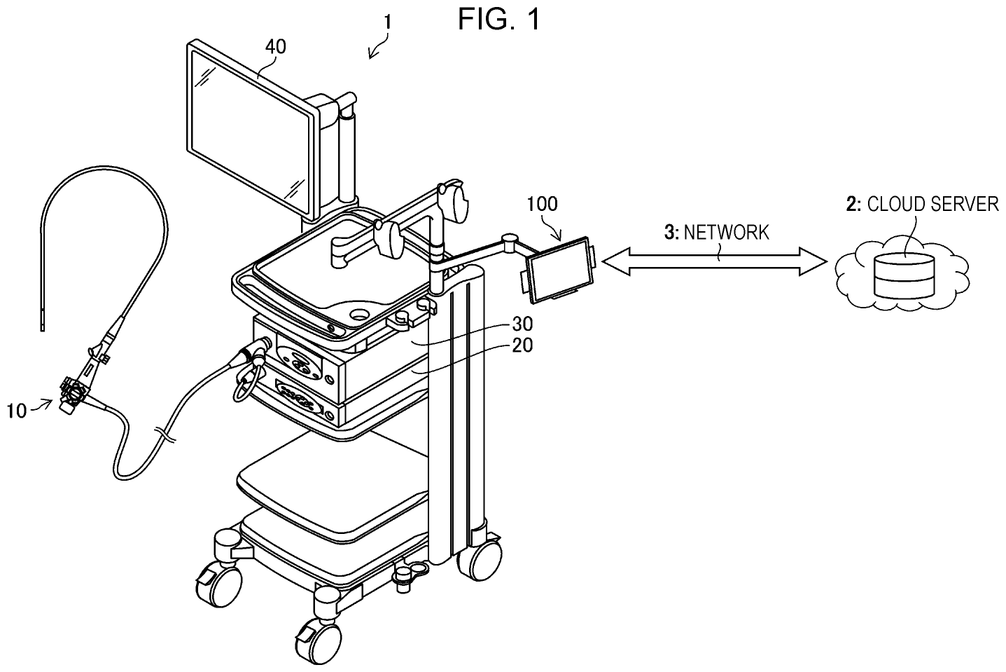

FIG. 1 is a system configuration diagram including a tablet terminal that functions as an information processing device and an endoscope system according to the present invention.

In FIG. 1, an endoscope system 1 includes an endoscope 10, a processor device 20, a light source device 30, and a first display device 40, to which a conventional system can be applied.

A tablet terminal 100 which functions as an information processing device is attached to a cart on which the endoscope system 1 is mounted. The tablet terminal 100 is connected to a cloud server (server) 2 through a network 3, and can download a speech recognition engine from the cloud server 2 as described later.

### Processor Device

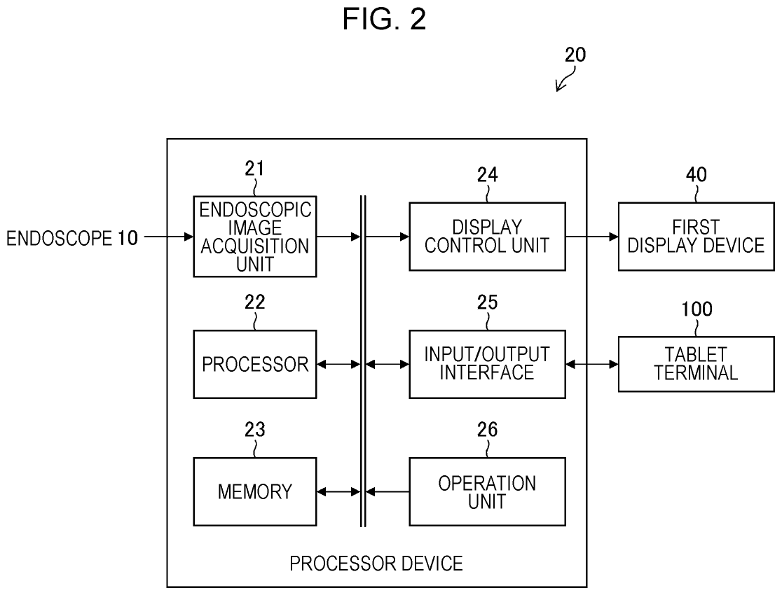

FIG. 2 is a block diagram illustrating an embodiment of a hardware configuration of a processor device that forms the endoscope system illustrated in FIG. 1.

The processor device 20 illustrated in FIG. 2 includes an endoscopic image acquisition unit 21, a processor 22, a memory 23, a display control unit 24, an input/output interface 25, and an operation unit 26.

The endoscopic image acquisition unit 21 includes a connector to which the endoscope 10 is connected, and acquires, from the endoscope 10 through the connector, an endoscopic image (dynamic image) picked up by an imaging device located at the distal end portion of the endoscope 10. Also, the processor device 20 acquires, through the connector to which the endoscope 10 is connected, a remote signal in response to an operation performed using an operation unit for manipulating the endoscope 10. The remote signal includes a release signal giving an instruction to take a still image, an observation mode switch signal for switching observation modes, and the like.

The processor 22 includes a central processing unit (CPU) or the like that centrally controls each unit of the processor device 20 and functions as a processing unit that performs processing, such as image processing of an endoscopic image acquired from the endoscope 10, artificial intelligence (AI) processing to recognize lesions from endoscopic images in real time, and processing for acquiring and saving still images according to the release signal acquired through the endoscope 10.

The memory 23 includes flash memory, read-only memory (ROM) and random access memory (RAM), a hard disk apparatus, and the like. The flash memory, ROM, or hard disk apparatus is a non-volatile memory storing various programs or the like to be executed by the processor 22. The RAM functions as a work area for processing by the processor 22, and also temporarily stores programs or the like stored in the flash memory or the like. Note that the processor 22 may incorporate a portion (the RAM) of the memory 23. Still images taken during endoscopy can be saved in the memory 23.

The display control unit 24 generates an image for display on the basis of a real-time endoscopic image (dynamic image) and still images that have been subjected to image processing by the processor 22 and various information (for example, information about a lesion area, information about the area under observation, and the state of speech recognition) processed by the processor 22, and outputs the image for display to the first display device 40.

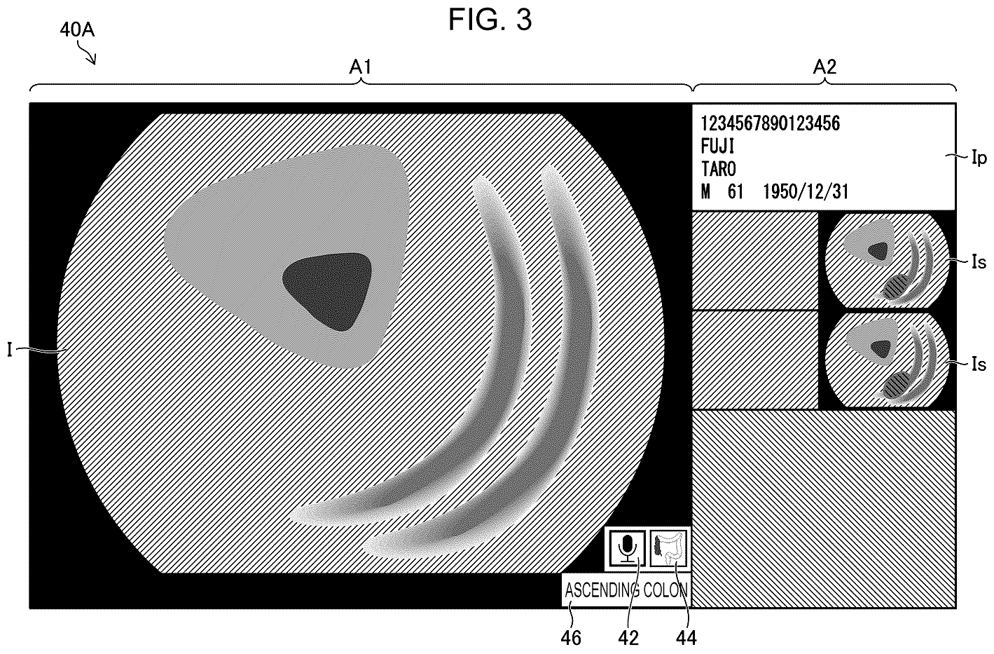

FIG. 3 is a diagram illustrating an example of a display screen on a first display device that forms the endoscope system illustrated in FIG. 1.

As illustrated in FIG. 3, a screen 40A of the first display device 40 has a main display area A1 and a sub display area A2. In the main display area A1, an endoscopic image I (dynamic image) is displayed. Also, when a lesion is recognized by the processor 22, a bounding box or the like enclosing the area of the lesion is displayed to support image diagnosis.

In the sub display area A2 of the screen 40A, various information related to endoscopy is displayed. In the example illustrated in FIG. 3, patient-related information Ip and still images Is of endoscopic images taken during endoscopy are displayed. The still images Is are displayed from top to bottom on the screen 40A in the order in which the images were taken, for example.

Additionally, the processor 22 can display an icon 42 indicating the state of speech recognition to be described later, a typical diagram (schema diagram) 44 illustrating the area under observation during image-taking, and the name 46 of the area under observation (in this example, the ascending colon) on the screen 40A of the first display device 40.

Returning to FIG. 2, the input/output interface 25 includes a connection unit for establishing a wired and/or wireless connection with external equipment, a communication unit capable of communicating with a network, and the like. In this example, the processor device 20 is wirelessly connected to the tablet terminal 100 through the input/output interface 25, and transmits and receives necessary information.

Additionally, foot switches not illustrated are connected to the input/output interface 25. The foot switches are operating devices placed at the feet of the operator and operated by the feet, and an operation signal is transmitted to the processor device 20 by depressing a pedal. The processor device 20 is connected to storage, not illustrated, through the input/output interface 25. The storage not illustrated is an external storage device connected to the processor device 20 by a local area network (LAN) or the like, and is a file server of a picture archiving and communication system (PACS) or other system for filing endoscopic images, or network-attached storage (NAS), for example.

The operation unit 26 includes a power switch, switches for manually adjusting parameters such as white balance, light intensity, and zooming, switches for setting various modes, and the like.

The light source device 30 is connected to the endoscope 10 through a connector, and thereby supplies illumination light to a light guide of the endoscope 10. The illumination light is selected from light in various wavelength ranges according to the purpose of observation, such as white light (light in the white wavelength range or light in multiple wavelength ranges), light in one or more specific wavelength ranges, or a combination of these. Note that a specific wavelength range is a narrower range than the white wavelength range. Light in various wavelength ranges can be selected by a switch for selecting the observation mode.

### Hardware Configuration of Tablet Terminal

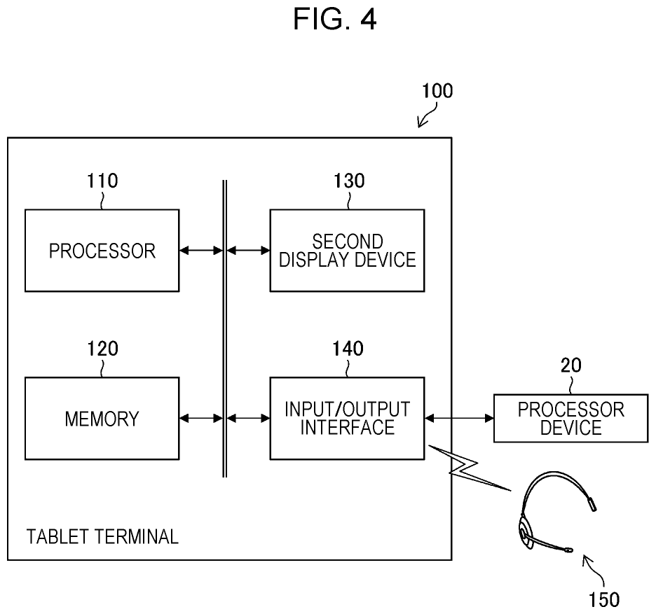

FIG. 4 is a block diagram illustrating an embodiment of a hardware configuration of the tablet terminal illustrated in FIG. 1.

The tablet terminal 100 illustrated in FIG. 4 includes a processor 110, a memory 120, a second display device 130, and an input/output interface 140.

The processor 110 includes a CPU or the like that centrally controls each unit of the tablet terminal 100 and functions as a processing unit that recognizes speech uttered by the user during endoscopy and a processing unit that acquires record information to be recorded in relation to endoscopy on the basis of speech recognition results.

The memory 120 includes flash memory, read-only memory (ROM) and random access memory (RAM), a hard disk apparatus, and the like. The flash memory, ROM, or hard disk apparatus is a non-volatile memory storing various programs to be executed by the processor 110, such as an information processing program according to the present invention and a speech recognition engine, a first dictionary according to the present invention, and the like. The RAM functions as a work area for processing by the processor 110, and also temporarily stores programs or the like stored in the flash memory or the like. Note that the processor 110 may incorporate a portion (the RAM) of the memory 120. Also, endoscopic images (still images) taken during endoscopy and record information acquired by the processor 110 can be saved in the memory 120.

The second display device 130 is a display with a touch panel and functions as a graphical user interface (GUI) for displaying speech recognition results recognized by the processor 110, record information acquired by the processor 110, the first dictionary, and the like, and accepting various instructions and information according to touches on the screen.

The input/output interface 140 includes a connection unit for establishing a wired and/or wireless connection with external equipment, a communication unit capable of communicating with a network, and the like. In this example, the tablet terminal 100 is wirelessly connected to the processor device 20 through the input/output interface 140, and transmits and receives necessary information.

A microphone 150 is connected to the input/output interface 140, and the input/output interface 140 receives voice data from the microphone 150. Note that the microphone 150 in this example is a wireless headset placed on the head of the user (physician), and transmits voice data representing speech uttered by the user during endoscopy.

The tablet terminal 100 is connected to the cloud server 2 through the network 3 as illustrated in FIG. 1, with the communication unit of the input/output interface 140 being capable of connecting to the network 3.

Note that, preferably, the tablet terminal 100 is attached to a cart or the like such that only the user can see the screen of the tablet terminal 100. On the other hand, the first display device 40 of the endoscope system 1 may be installed so that the both the user and the patient can see the screen.

### First Embodiment of Tablet Terminal

When performing endoscopy, the user (physician) operates the endoscope 10 with both hands, moves the distal end of the scope to a desired area inside a luminal organ of a photographic subject, and takes an endoscopic image (dynamic image) using the imaging device located at the distal end portion of the scope. The endoscopic image taken by the endoscope 10 undergoes image processing by the processor device 20 and then is displayed in the main display area A1 of the screen 40A of the first display device 40, as illustrated in FIG. 3.

During endoscopy, the user performs operations such as advancing and retracting the distal end of the scope while checking the endoscopic image (dynamic image) displayed on the screen 40A of the first display device 40. Upon discovering a lesion or the like in the area under observation inside a luminal organ, the user takes a still image of the area under observation by operating a release button for giving an instruction to take a still image, and also makes a diagnosis, applies treatment using the endoscope, and the like. Note that the processor device 20 can provide diagnostic support by performing AI processing or the like to recognize lesions from endoscopic images in real time as described above.

The tablet terminal 100 is a piece of equipment for acquiring record information to be recorded in relation to endoscopy on the basis of speech uttered by the user and recording the acquired record information in association with a still image during endoscopy as above.

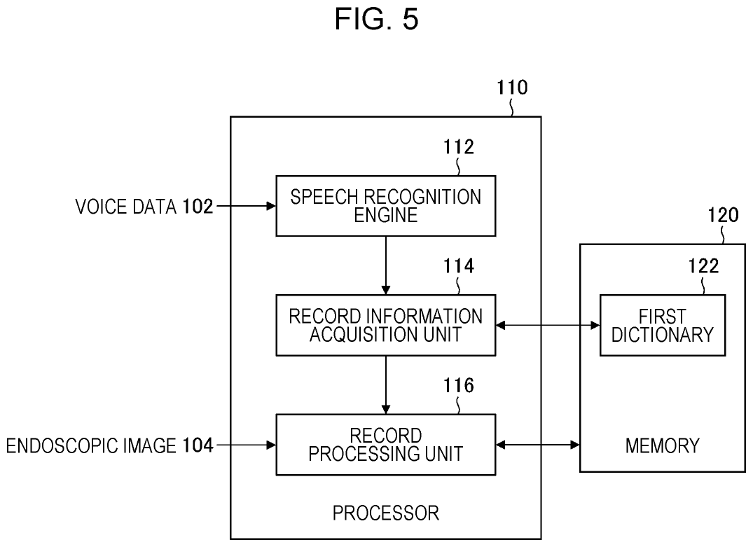

FIG. 5 is a functional block diagram illustrating a first embodiment of a tablet terminal and illustrating the processor 110 in particular.

As illustrated in FIG. 5, the processor 110 executes an information processing program and a speech recognition engine stored in the memory 120, and thereby functions as a speech recognition unit using a speech recognition engine 112, as a record information acquisition unit 114, and as a record processing unit 116.

When the user discovers a lesion during endoscopy, the user takes an endoscopic image (still image) showing the lesion and utters speech expressing identifying characters that differ from the record information to be recorded in association with the endoscopic image (such as the name of a diagnosis, the name of a treatment using the endoscope, and the name of the treatment tool used in the treatment, for example).

The microphone 150 of the headset converts speech uttered by the user into an electrical signal (voice data). The voice data 102 is received by the input/output interface 140 and input into the processor 110.

The processor 110 uses the speech recognition engine 112 to convert the voice data representing identifying characters corresponding to record information into identifying characters (text data). That is, the processor 110 recognizes user-uttered speech expressing identifying characters.

On the basis of the identifying characters that the speech recognition engine 112 has obtained by speech recognition, the record information acquisition unit 114 acquires (reads out) record information corresponding to the identifying characters from a first dictionary 122 in the memory 120.

### First Dictionary

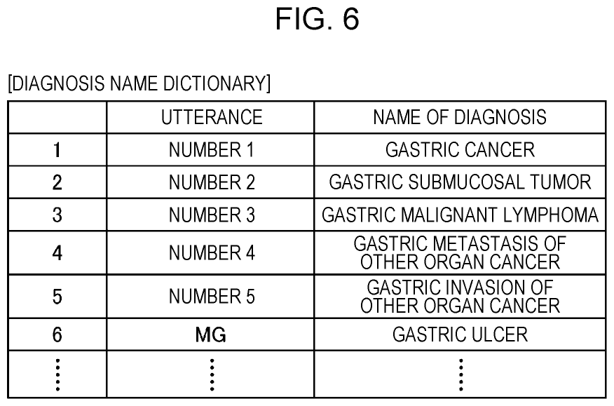

FIG. 6 is a diagram illustrating an example of a diagnosis name dictionary which is a first dictionary saved in a memory of a tablet terminal.

The first dictionary 122 illustrated in FIG. 6 is a diagnosis name dictionary containing the names of diagnoses indicating lesions as record information, in which identifying characters to be uttered and the names of diagnoses are associated with each other.

The identifying characters to be uttered are numerals such as Number 1, Number 2, Number 3, and so on, and the abbreviation MG (Magen Geschwuer) for the name of the diagnosis of gastric ulcer, these being different from the names of diagnoses which are record information.

In this way, in the diagnosis name dictionary, which is the first dictionary 122, identifying characters that differ from the names of diagnoses that the patient would be afraid to hear are associated with each name of a diagnosis.

In this example, when recording the name of a diagnosis by voice operation, instead of uttering the name of the diagnosis, the user utters the number associated with the name of the diagnosis or the abbreviation for the name of the diagnosis.

Note that the identifying characters that differ from the names of diagnoses are not limited to numerals such as numbers and abbreviations for the names of diagnoses, and individual letters of the alphabet, individual letters of the alphabet combined with numerals, or the like may also be considered. In short, the identifying characters may be any identifying characters that would not remind the patient of the names of diagnoses. Also, in the case of adopting an abbreviation for the name of a diagnosis as identifying characters, the identifying characters are preferably an abbreviation for the name of a diagnosis which is not a serious illness.

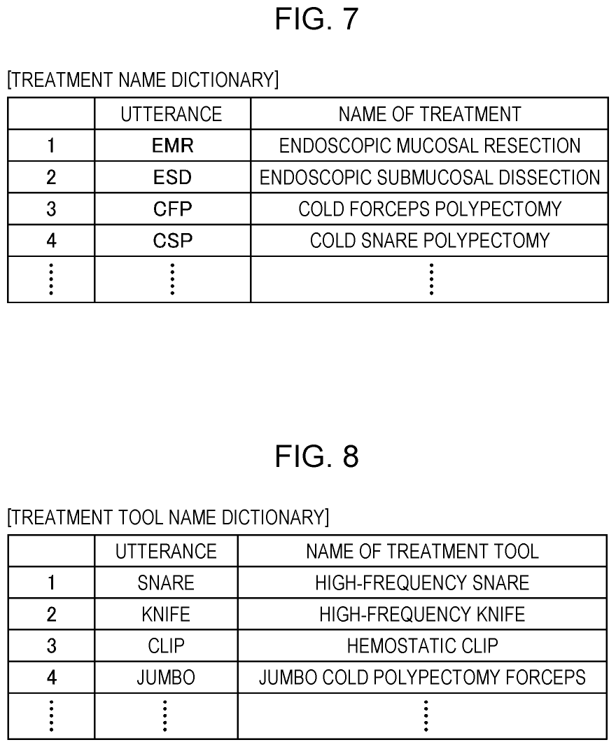

FIG. 7 is a diagram illustrating an example of a treatment name dictionary which is a first dictionary saved in a memory of a tablet terminal.

The first dictionary 122 illustrated in FIG. 7 is a treatment name dictionary containing the names of treatments indicating treatments involving an endoscope as record information, in which identifying characters to be uttered and the names of treatments are associated with each other.

In this case, the identifying characters to be uttered are abbreviations for the names of treatments involving an endoscope, such as endoscopic mucosal resection (EMR), endoscopic submucosal dissection (ESD), cold forceps polypectomy (CFP), and cold snare polypectomy (CSP).

The official names of treatments involving an endoscope may be long names in some cases, while on the other hand, the user is accustomed to using the abbreviations for these names of treatments. Accordingly, the abbreviations for the names of treatments are suitable as the identifying characters to be uttered.

FIG. 8 is a diagram illustrating an example of a treatment tool name dictionary which is a first dictionary saved in a memory of a tablet terminal.

The first dictionary 122 illustrated in FIG. 8 is a treatment tool name dictionary containing treatment tools used in treatments involving an endoscope as record information, in which identifying characters to be uttered and the names of treatment tools are associated with each other.

In this case, the identifying characters to be uttered are abbreviations or common names for the names of treatment tools such as high-frequency snare, high-frequency knife, hemostatic clip, and jumbo cold polypectomy forceps. The official names of treatment tools may be long names in some cases, while on the other hand, the user is accustomed to using the abbreviations or common names for these names of treatment tools. Accordingly, the abbreviations or common names for the names of treatment tools are suitable as the identifying characters to be uttered.

Returning to FIG. 5, when a still image is taken during endoscopy, the record processing unit 116 acquires the still image as an endoscopic image 104 from the processor device 20, and when the record information acquisition unit 114 acquires record information corresponding to identifying characters from the first dictionary 122 on the basis of identifying characters provided by voice operation during endoscopy, the record processing unit 116 saves the acquired endoscopic image 104 and the record information in association with each other in the memory 120. The endoscopic image and the record information saved in the memory 120 can be used to create a diagnostic report, for example.

### Second Embodiment of Tablet Terminal

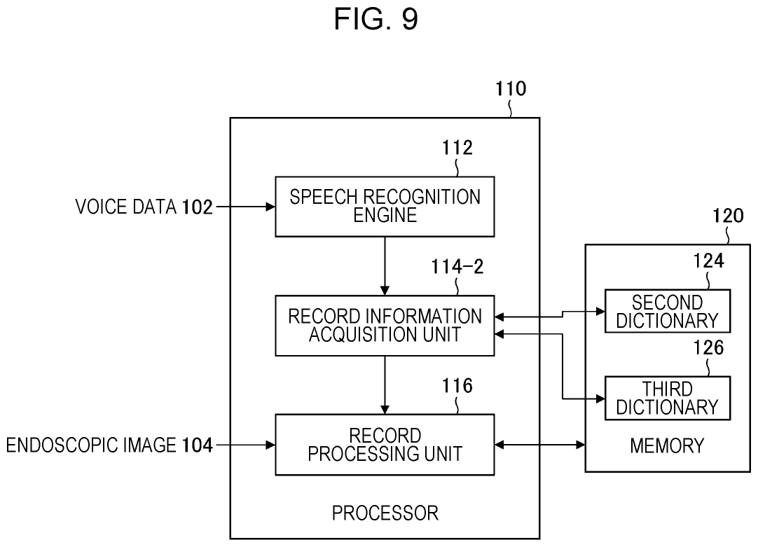

FIG. 9 is a functional block diagram illustrating a second embodiment of a tablet terminal and illustrating the processor 110 in particular. Note that in FIG. 9, portions in common with the tablet terminal according to the first embodiment illustrated in FIG. 5 are denoted with the same signs, and a detailed description of such portions is omitted.

The tablet terminal according to the second embodiment illustrated in FIG. 9 mainly differs in that the tablet terminal according to the second embodiment uses a second dictionary 124 and a third dictionary 126 instead of the first dictionary 122 in the tablet terminal according to the first embodiment. That is, the first dictionary 122 is formed from the second dictionary 124 and the third dictionary 126.

In the second dictionary 124, identification information indicating record information and record information are registered in association with each other, and in the third dictionary 126, identifying characters and identification information are registered in association with each other. The second dictionary 124 and the third dictionary 126 serve similarly to the first dictionary 122.

A record information acquisition unit 114-2 of the processor 110 acquires identification information corresponding to identifying characters from the third dictionary 126 in the memory 120 on the basis of the identifying characters that the speech recognition engine 112 has obtained by speech recognition, and subsequently acquires record information associated with the identification information from the second dictionary 124 on the basis of the acquired identification information.

The first dictionary 122 is configured such that record information and identifying characters that differ from the record information are associated with each other directly, but in the case where the first dictionary 122 is formed from the second dictionary 124 and the third dictionary 126, record information and identifying characters that differ from the record information are associated with each other indirectly through identification information.

### Second Dictionary and Third Dictionary

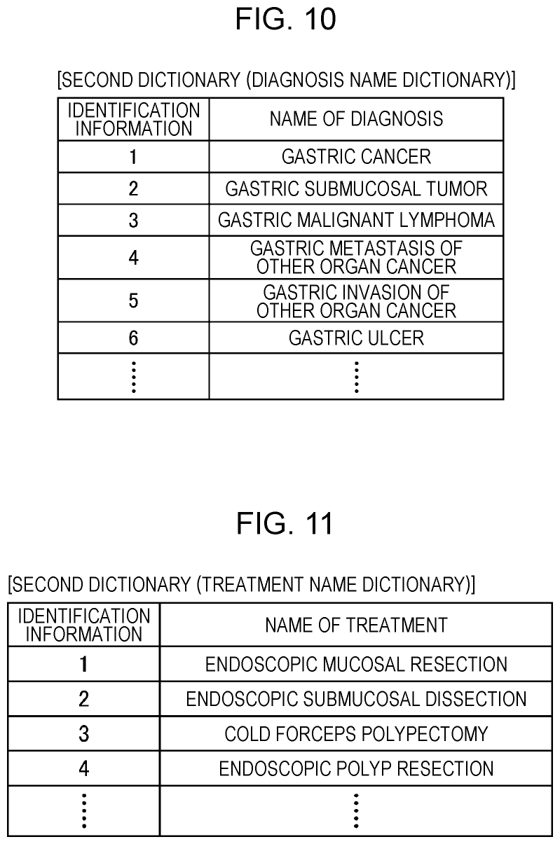

FIG. 10 is a diagram illustrating an example of a diagnosis name dictionary which is a second dictionary saved in a memory of a tablet terminal.

The diagnosis name dictionary which is the second dictionary 124 illustrated in FIG. 10 is a dictionary containing names of diagnoses indicating lesions as record information. The names of all diagnoses that are diagnosed during endoscopy are registered in this diagnosis name dictionary, and the identification information specifying each name of a diagnosis can be, for example, diagnosis name dictionary plus a serial number.

FIG. 11 is a diagram illustrating an example of a treatment name dictionary which is a second dictionary saved in a memory of a tablet terminal.

The treatment name dictionary which is the second dictionary 124 illustrated in FIG. 11 is a dictionary containing names of treatments indicating treatments involving an endoscope as record information. The names of treatments indicating all treatments that are performed during endoscopy are registered in this treatment name dictionary, and the identification information specifying each name of a treatment can be, for example, treatment name dictionary plus a serial number.

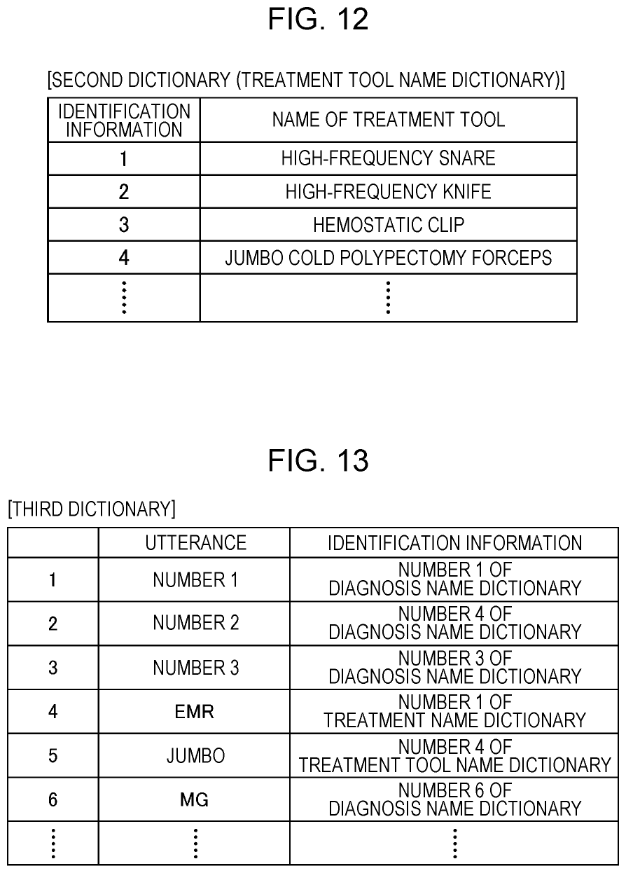

FIG. 12 is a diagram illustrating an example of a treatment tool name dictionary which is a second dictionary saved in a memory of a tablet terminal.

The treatment tool name dictionary which is the second dictionary 124 illustrated in FIG. 12 is a dictionary containing names of treatment tools indicating treatment tools used in treatments involving an endoscope as record information. The names of treatment tools indicating all treatment tools that are used in treatments involving an endoscope are registered in this treatment tool name dictionary, and the identification information specifying each name of a treatment tool can be, for example, treatment tool name dictionary plus a serial number.

FIG. 13 is a diagram illustrating an example of a third dictionary saved in a memory of a tablet terminal.

In the third dictionary 126 illustrated in FIG. 13, identifying characters to be uttered by the user and identification information are registered in association with each other.

According to the third dictionary 126 illustrated in FIG. 13, when the identifying characters uttered by the user are “Number 1” (when the speech recognition engine 112 recognizes “Number 1”), the record information acquisition unit 114-2 illustrated in FIG. 9 acquires “Number 1 of diagnosis name dictionary” as the identification information associated with “Number 1” from the third dictionary 126. Additionally, from the acquired identification information “Number 1 of diagnosis name dictionary”, “Gastric cancer” is acquired as the name of the diagnosis, because “Gastric cancer” is the name of the diagnosis for “Number 1” in the diagnosis name dictionary which is the second dictionary illustrated in FIG. 10.

Similarly, according to the third dictionary 126 illustrated in FIG. 13, when the identifying characters uttered by the user are “EMR”, “Number 1 of treatment name dictionary” is acquired as the identification information associated with “EMR” from the third dictionary 126. Additionally, from the acquired identification information “Number 1 of treatment name dictionary”, “Endoscopic mucosal resection” is acquired as the name of the treatment, because “Endoscopic mucosal resection” is the name of the treatment for “Number 1” in the diagnosis name dictionary which is the second dictionary illustrated in FIG. 11.

### Creation of Third Dictionary

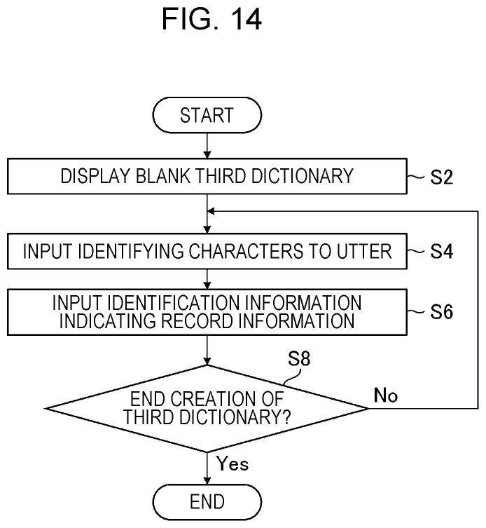

FIG. 14 is a flowchart illustrating a procedure for using a tablet terminal to create a third dictionary.

The user can newly create the third dictionary 126 by operation input using the GUI of the tablet terminal 100. In this case, the function of the tablet terminal 100 for creating the third dictionary 126 first causes the second display device 130 to display a blank third dictionary (step S2).

Next, desired identifying characters (“Number 1”, for example) for the user to utter are inputted into a field for inputting identifying characters in the blank third dictionary (step S4).

The user inputs desired identification information (“Number 1 of diagnosis name dictionary”, for example) into an identification information field corresponding to the inputted identifying characters (step S6). Note that this assumes the user can check the content of the second dictionary (diagnosis name dictionary) on the screen of the tablet terminal 100 or the like.

After inputting pairs of identifying characters and identification information in this way, the user determines whether or not to end creation of the third dictionary (step S8).

In the case of not ending creation of the third dictionary, the user continues to repeat the input in steps S4 and S6 and creates the third dictionary.

The user can choose to end creation of the third dictionary to complete and save the third dictionary 126 in the memory 120.

Note that the user can also edit the third dictionary 126 (add, change, or remove pairs of identifying characters and identification information) in a similar way.

Moreover, the third dictionary 126 can be saved in the memory 120 as custom user dictionaries (multiple dictionaries) for multiple users. In this case, the second dictionary 124 can be used in common among the multiple users.

### Setting First Dictionary to Enabled/Disabled and Operating Method for Information Processing Device

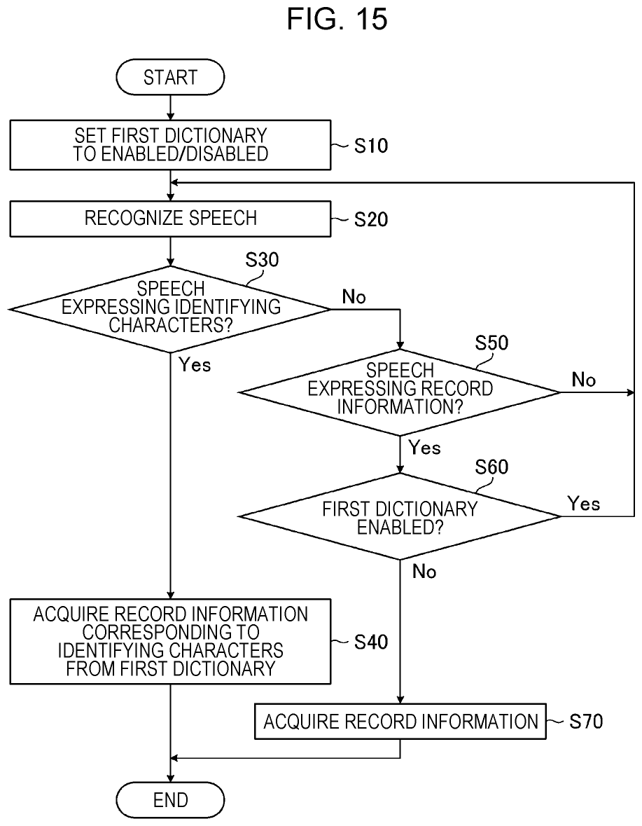

FIG. 15 is a flowchart illustrating the flow of setting a first dictionary to enabled/disabled and acquiring record information according to an operating method for an information processing device in a tablet terminal.

In FIG. 15, the first dictionary is set to enabled/disabled (step S10). The first dictionary may be set to enabled/disabled by the user by operation input from the GUI of the tablet terminal 100, or automatically as described later.

The first dictionary includes the first dictionary 122 illustrated in FIG. 5 and a dictionary that functions as a first dictionary formed from the second dictionary 124 and third dictionary 126 illustrated in FIG. 9.

In setting the first dictionary to enabled/disabled, the “enabled” setting refers to a setting in which record information, such as the name of a diagnosis, is acquired by voice operation with the use of the first dictionary, whereas the “disabled” setting refers to a setting in which record information, such as the name of a diagnosis, is acquired by voice operation with or without the use of the first dictionary.

The processor 110 uses the speech recognition engine 112 to recognize speech uttered by the user during endoscopy (step S20).

Next, the processor 110 determines whether or not the recognized speech expresses identifying characters registered in the first dictionary (step S30). If it is determined that the recognized speech expresses identifying characters (the “Yes” case), the processor 110 acquires record information corresponding to the identifying characters from the first dictionary (step S40).

This allows the user to utter identifying characters different from the name of a diagnosis that the patient would be afraid to hear, and thereby acquire the name of the diagnosis (record information) corresponding to the identification information. This also allows the user to utter an abbreviation or the like that the user is accustomed to using for the name of a treatment involving an endoscope, and thereby acquire the official name of the treatment (record information) corresponding to the identification information.

On the other hand, in step S30, if it is determined that the recognized speech is not speech expressing identifying characters (the “No” case), the processor 110 further determines whether or not the recognized speech is speech expressing record information such as the name of a diagnosis to be recorded during endoscopy (step S50). If it is determined that the recognized speech is not record information, the processor 110 proceeds to step S20 and the recognized speech is not acquired as record information. If it is determined that the recognized speech is record information, the processor 110 proceeds to step S60.

In step S60, the processor 110 determines whether or not the first dictionary is set to enabled. If it is determined that the first dictionary is set to enabled (the “Yes” case), the processor 110 proceeds to step S20. Accordingly, even if the recognized speech is record information, that record information is not acquired. This is because when the first dictionary is set to enabled, only the acquisition of record information through the utterance of identifying characters with the use of the first dictionary is allowed.

On the other hand, if it is determined in step S60 that the first dictionary is set to disabled (the “No” case), the processor 110 proceeds to step S70 and acquires the record information that has been uttered at this point. Consequently, when the first dictionary is set to disabled, record information can be acquired through the utterance of identifying characters with the use of the first dictionary, and record information can also be acquired when the record information is uttered directly.

### Automatically Setting First Dictionary to Enabled/Disabled

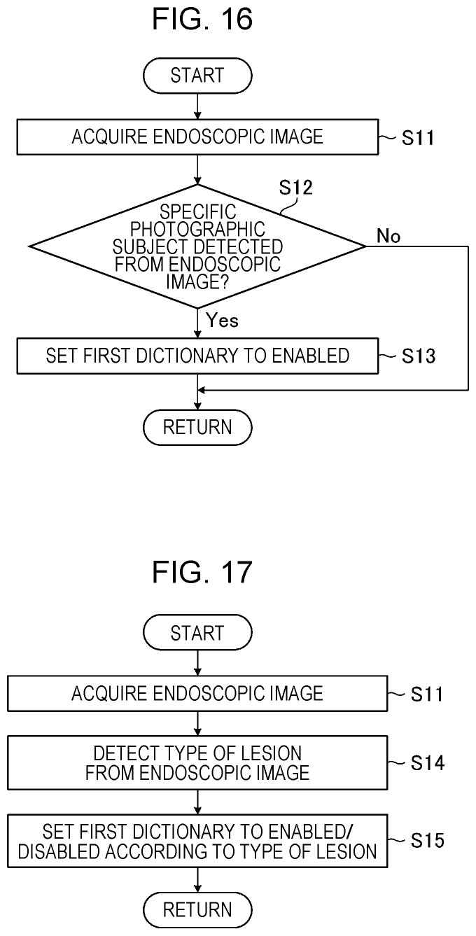

FIG. 16 is a flowchart illustrating an example of automatically setting a first dictionary to enabled/disabled in a tablet terminal, and illustrates an example of the processing in step S10 illustrated in FIG. 15.

In FIG. 16, the processor 110 of the tablet terminal 100 acquires an endoscopic image during endoscopy (step S11) and determines whether or not a specific type of photographic subject is detected from the acquired endoscopic image (step S12). The specific type of photographic subject is a lesion, and can be a photographic subject indicative of “neoplastic” out of neoplastic/non-neoplastic, for example. Note that neoplastic/non-neoplastic can be recognized from endoscopic images by AI.

If it is determined that the specific type of photographic subject is detected (the “Yes” case), the processor 110 sets the first dictionary to enabled (step S13). On the other hand, if the specific type of photographic subject is not detected (the “No” case), the first dictionary is not set to enabled (is set to disabled).

In this way, when the specific type of photographic subject is detected, the first dictionary is automatically set to enabled, and as a result, the acquisition of record information is limited to the case of acquisition through the utterance of identifying characters with the use of the first dictionary. For example, in the case where the specific type of photographic subject (for example, a neoplastic lesion) is detected, the first dictionary can be enabled so that record information cannot be acquired through the utterance of words that the patient would be afraid to hear (the names of diagnoses related to neoplasms).

FIG. 17 is a flowchart illustrating another example of automatically setting a first dictionary to enabled/disabled in a tablet terminal, and illustrates another example of the processing in step S10 illustrated in FIG. 15.

In FIG. 17, the processor 110 of the tablet terminal 100 acquires an endoscopic image during endoscopy (step S11) and detects the type of lesion from the acquired endoscopic image (step S14). The type of lesion is not limited to neoplastic/non-neoplastic, and includes a plurality of types of lesions corresponding to the plurality of names of diagnoses registered in the diagnosis name dictionary, for example. Also, types of lesions can be recognized from endoscopic images by a lesion recognition AI.

The processor 110 automatically sets the first dictionary to enabled or disabled according to the type of lesion detected (step S15). The types of lesions for which to enable the first dictionary can be set in advance. For example, the first dictionary can be set to enabled for lesions of serious illnesses that the patient would be afraid to hear.

Consequently, when a specific lesion (a lesion for which to enable the first dictionary) is detected from an endoscopic image, the first dictionary is automatically set to enabled for that specific lesion. This means that, for example, when a lesion of a serious illness that the patient would be afraid to hear is detected, the name of the diagnosis of the lesion is acquired by voice operation by uttering identifying characters that differ from the name of the diagnosis to acquire the name of the diagnosis from the first dictionary.

Note that in the automatic setting of the first dictionary to enabled/disabled illustrated in FIGS. 16 and 17, the process to detect the specific photographic subject from the endoscopic image and the process to detect the type of lesion from the endoscopic image are not limited to being performed by the processor 110 of the tablet terminal 100. The processor device 20 may perform these processes and transmit the detection results to the tablet terminal 100.

### Downloading Speech Recognition Engine

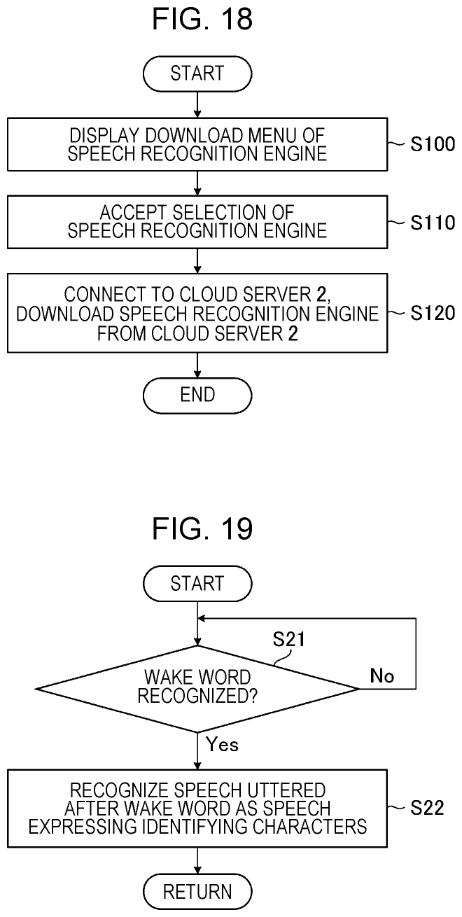

FIG. 18 is a flowchart illustrating a procedure by which a tablet terminal acquires a speech recognition engine.

The tablet terminal 100 can download a speech recognition engine provided by the cloud server 2 illustrated in FIG. 1. In this case, a plurality of speech recognition engines are prepared in the cloud server 2, and the user is able to download a desired speech recognition engine from among the plurality of speech recognition engines.

In FIG. 18, in the case of downloading a speech recognition engine, the user operates the tablet terminal 100 to display a menu screen for downloading a speech recognition engine (step S100). Preferably, for example, an input field is displayed on the menu screen to allow for the input of user attributes or the like.

The tablet terminal 100 accepts the selection of a speech recognition engine from the user on the basis of an operation performed by the user on the menu screen (step S110). For example, the user follows the menu screen and inputs user attributes (language used, gender, age, geographical region) or the like, whereby the tablet terminal 100 accepts the selection of a speech recognition engine suited to that user. Inputting a language used allows for the selection of a speech recognition engine for Japanese, English, or other language, while inputting a gender and an age allows for the selection of a speech recognition engine suited to recognizing speech by a person of the corresponding gender and age. Inputting a geographical region allows for the selection of a speech recognition engine suited to the intonation of speech used in that geographical region.

Upon accepting the selection of a speech recognition engine, the tablet terminal 100 connects to the cloud server 2 and downloads the selected speech recognition engine from the cloud server 2 (step S120).

This eliminates the need to prepare a speech recognition engine in advance on the tablet terminal side and allows for the acquisition of a speech recognition engine suited to the attributes of the user. Note that when the latest speech recognition engine is developed on the cloud server 2 side, the user is notified by the cloud server 2, and the user can update a speech recognition engine to the latest speech recognition engine.

### Utilization of Wake Word

FIG. 19 is a flowchart illustrating an example of utilizing speech recognition of a wake word.

For example, in step S20 illustrated in FIG. 15, if speech expressing a wake word is recognized during endoscopy, the tablet terminal 100 is triggered by the speech recognition of the wake word to start the recognition of speech expressing identifying characters or the like uttered thereafter. Note that the wake word is assumed to be set in advance in the speech recognition engine.

The processor 110 of the tablet terminal 100 determines whether or not characters that the speech recognition engine has obtained by speech recognition are a wake word (step S21). If the characters are determined to be a wake word (the “Yes” case), the processor 110 uses the speech recognition engine to recognize speech uttered after the wake word, and acquires the result of the recognition as identifying characters (step S22).

Identifying characters are assumed to be short words or phrases that may be uttered in situations where the user does not intend identifying characters to be uttered, but by using a wake word as a trigger to recognize speech of identifying characters, the identifying characters can be recognized with greater accuracy.

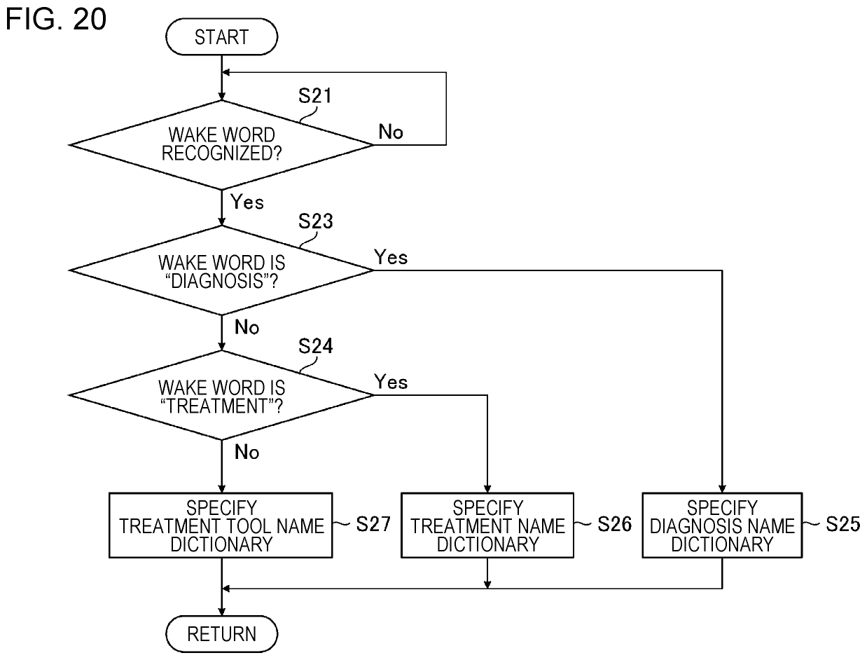

FIG. 20 is a flowchart illustrating another example of utilizing speech recognition of a wake word.

As the wake word in this example, a plurality of wake words are set, such as “Diagnosis”, “Treatment”, and “Treatment tool”, for example.

In FIG. 20, the processor 110 of the tablet terminal 100 determines whether or not characters that the speech recognition engine has obtained by speech recognition are a wake word (step S21). If the characters are determined to be a wake word (the “Yes” case), the processor 110 determines whether or not the wake word indicates “Diagnosis”, and whether or not the wake word indicates “Treatment” (steps S23, S24).

If the wake word is determined to be “Diagnosis”, the processor 110 specifies the diagnosis name dictionary (step S25). If the wake word is determined to be “Treatment”, the processor 110 specifies the treatment name dictionary (step S26). If the wake word is determined to be other than “Diagnosis” or “Treatment” (that is, “Treatment tool”), the processor 110 specifies the treatment tool name dictionary (step S27).

The processor 110 can acquire record information corresponding to identifying characters from the dictionary specified by the wake word on the basis of identifying characters recognized from an utterance after the wake word.

The tablet terminal 100 is triggered by speech recognition of a wake word to start the recognition of speech expressing identifying characters or the like uttered thereafter, similarly to the case in FIG. 19, but is further configured to specify a dictionary according to the type of wake word. Thus, candidates of the identifying characters to obtain by speech recognition can be narrowed down to a specific dictionary, and misrecognition in speech recognition can be reduced.

Note that a wake word may be a word specifying at least one dictionary from among a diagnosis name dictionary, a treatment name dictionary, and a treatment tool name dictionary.

### Dictionary Selection

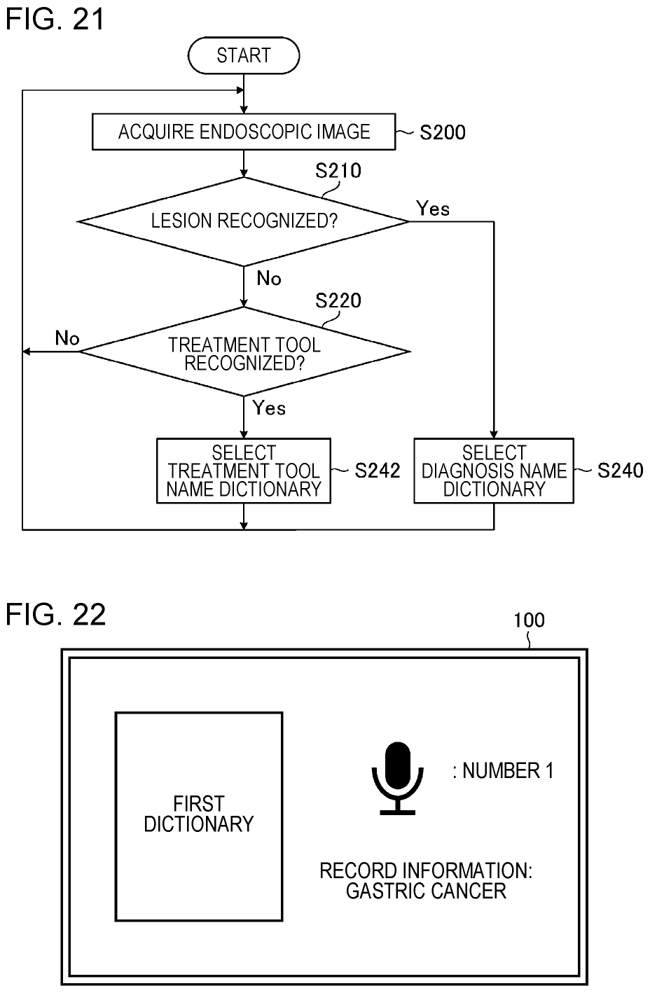

FIG. 21 is a flowchart illustrating an example of automatically selecting a diagnosis name dictionary and a treatment tool name dictionary.

In the example illustrated in FIG. 20, a dictionary is specified (selected) according to the type of wake word, but the automatic dictionary selection illustrated in FIG. 21 is based on an endoscopic image.

In FIG. 21, the processor 110 of the tablet terminal 100 acquires an endoscopic image (step S200). The processor 110 recognizes whether or not the acquired endoscopic image shows a lesion, or whether or not the acquired endoscopic image shows a treatment tool (steps S210, S220). Lesions and treatment tools can be recognized from endoscopic images by AI recognition.

If a lesion is recognized from the endoscopic image, the processor 110 selects the diagnosis name dictionary (step S240), whereas if a treatment tool is recognized from the endoscopic image, the processor 110 selects the treatment tool name dictionary (step S242).

The processor 110 can select the diagnosis name dictionary or the treatment tool name dictionary on the basis of a result of recognizing at least one of a lesion or a treatment tool, and acquire record information corresponding to identifying characters from the selected dictionary on the basis of recognized identifying characters. Note that when a treatment tool is recognized from an endoscopic image, the processor 110 may select the treatment tool name dictionary.

### Display of Dictionary and the Like

FIG. 22 is a diagram illustrating an example of a display screen on a tablet terminal during endoscopy.

If there is an unclear relationship between identifying characters to be uttered by the user and record information, such as the name of a diagnosis, corresponding to the identifying characters, the user will be unable to utter speech representing the corresponding identifying characters when acquiring desired record information.

The tablet terminal 100 illustrated in FIG. 22 displays the first dictionary on the display screen of the second display device 130 during endoscopy.

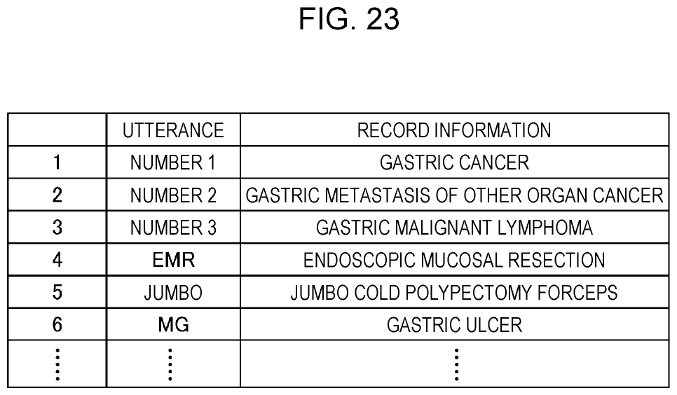

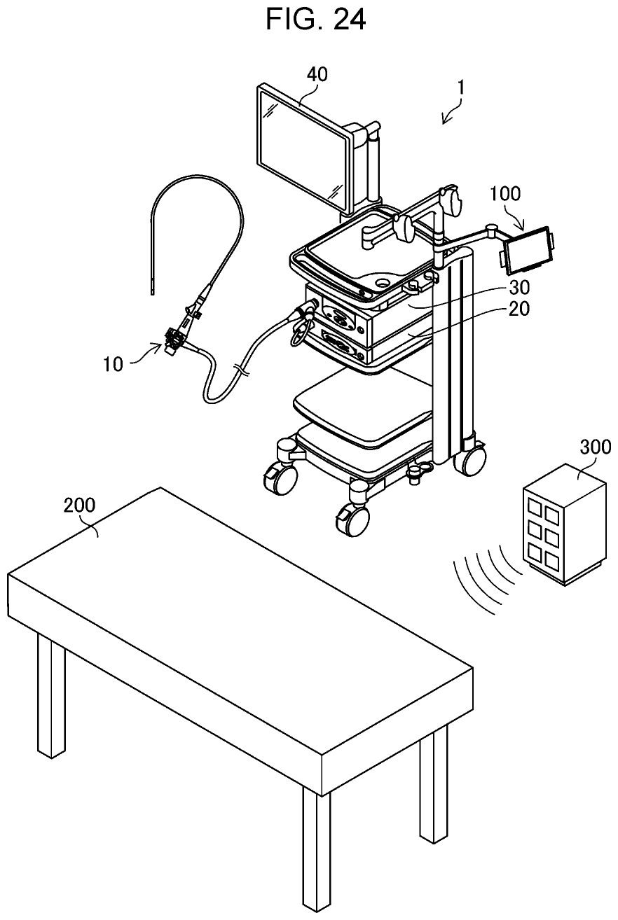

FIG. 23 is a diagram illustrating an example of a first dictionary displayed on the display screen in FIG. 22.

The first dictionary illustrated in FIG. 23 contains identifying characters to be uttered by the user and record information associated with the identifying characters. Also, the first dictionary illustrated in FIG. 23 contains a mix of names of diagnoses, names of treatments, and names of treatment tools, but may be configured as the three dictionaries of a diagnosis name dictionary, a treatment name dictionary, and a treatment tool name dictionary.

In the case where the first dictionary is configured as the three dictionaries of a diagnosis name dictionary, a treatment name dictionary, and a treatment tool name dictionary, the diagnosis name dictionary may be displayed on the second display device 130 of the tablet terminal 100, while the treatment name dictionary and the treatment tool name dictionary may be displayed in the sub display area A2 of the screen 40A of the first display device 40 of the endoscope system 1.

This is because, as described above, the tablet terminal 100 can be set up so that only the user (physician) can see the screen of the tablet terminal 100, and therefore even if the diagnosis name dictionary is displayed on the tablet terminal 100, the patient will be unable to connect speech expressing identifying characters with the name of a diagnosis.

Also, in the case where a dictionary is specified from among the diagnosis name dictionary, the treatment name dictionary, and the treatment tool name dictionary as illustrated in FIG. 20, or in the case where the diagnosis name dictionary or the treatment tool name dictionary is selected as illustrated in FIG. 21, the specified or selected dictionary may be displayed on the tablet terminal 100.

Furthermore, the processor of the tablet terminal 100 can display on the second display device 130 at least one of a result of recognizing speech uttered by the user or acquired record information. In the example illustrated in FIG. 22, the result of recognizing speech is “Number 1”, and the record information associated with “Number 1” is “Gastric cancer”.

This allows the user to confirm whether or not the speech recognition engine has correctly recognized an utterance by the user through speech recognition, and also confirm the record information to be recorded in association with an endoscopic image during endoscopy.

After confirming the record information, the user can operate a foot switch to save the endoscopic image and the record information in association with each other in the memory 120.

### Masking Sound Generating Device

FIG. 24 is a diagram illustrating an example of an examination room in which a masking sound generating device is disposed.

In FIG. 24, 200 denotes a bed on which the patient lies during endoscopy, and 300 denotes a masking sound generating device.

The user (physician) speaks into the microphone 150 during endoscopy, while the masking sound generating device 300 generates masking sound that inhibits the ability of the patient to hear the speech uttered by the user during endoscopy.

The wireless headset microphone 150 is located near the user's mouth, and thus can detect the user's speech without being inhibited by the masking sound, even when the user speaks quietly.

The Speech Privacy System (VSP-1, VSP-2) by Yamaha Corporation can be used as the masking sound generating device 300.

The masking sound generating device 300 can generate masking sound during endoscopy and thereby prevent the patient from hearing, or make it difficult to hear, utterances by the physician, and can also generate, as the masking sound, ambient sound that relaxes the patient.

### Other

The present embodiment describes the case of using the tablet terminal 100 which is independent from the processor device 20 as an information processing device, but the processor device 20 may be provided with some or all of the functions of the tablet terminal 100 according to the present embodiment.

Moreover, the hardware structure that carries out the various types of control by an information processing device according to the present invention is any of various types of processors like the following. The various types of processors include: a central processing unit (CPU), which is a general-purpose processor that executes software (a program or programs) to function as any of various types of control units; a programmable logic device (PLD) whose circuit configuration is modifiable after fabrication, such as a field-programmable gate array (FPGA); and a dedicated electric circuit, which is a processor having a circuit configuration designed for the specific purpose of executing a specific process, such as an application-specific integrated circuit (ASIC).

A single control unit may be configured as any one of these various types of processors, or may be configured as two or more processors of the same or different types (such as multiple FPGAs, or a combination of a CPU and an FPGA, for example). Moreover, multiple control units may be configured as a single processor. A first example of configuring a plurality of control units as a single processor is a mode in which a single processor is configured as a combination of software and one or more CPUs, as typified by a computer such as a client or a server, and the processor functions as the plurality of control units. A second example of the above is a mode utilizing a processor in which the functions of an entire system, including the plurality of control units, are achieved on a single integrated circuit (IC) chip, as typified by a system on a chip (SoC). In this way, various types of control units are configured as a hardware structure by using one or more of the various types of processors indicated above.

The present invention also includes an information processing program that, by being installed in a computer, causes the computer to function as an information processing device according to the present invention, and a non-transitory and computer-readable recording medium in which the information processing program is recorded.

Furthermore, the present invention is not limited to the foregoing embodiments, and obviously a variety of modifications are possible within a scope that does not depart from the spirit of the present invention.

### REFERENCE SIGNS LIST

1 endoscope system
2 cloud server
3 network
10 endoscope
20 processor device
21 endoscopic image acquisition unit
22 processor
23 memory
24 display control unit
25 input/output interface
26 operation unit
30 light source device
40 first display device
40A screen
42 icon
100 tablet terminal
102 voice data
104 endoscopic image
110 processor
112 speech recognition engine
114, 114-2 record information acquisition unit
116 record processing unit
120 memory
122 first dictionary
124 second dictionary
126 third dictionary
130 second display device
140 input/output interface
150 microphone
200 bed
300 masking sound generating device
A1 main display area
A2 sub display area
AI lesion recognition
I endoscopic image
Ip information
Is still image
S2-S8, S10-S70, S100-S120, S200-S240 step

What is claimed is:

1. An information processing device comprising
a processor and
a first dictionary in which record information to be recorded in relation to endoscopy is registered, wherein
the first dictionary is configured such that identifying characters that differ from the record information and the record information are associated directly or indirectly, and
the processor:
recognizes speech which is uttered by a user during endoscopy and which expresses the identifying characters; and
acquires the record information corresponding to the identifying characters from the first dictionary on a basis of the recognized identifying characters.

2. The information processing device according to claim 1, wherein the processor:
acquires an endoscopic image related to the record information during the endoscopy; and
saves the acquired endoscopic image and the record information in association with each other in a memory.

3. The information processing device according to claim 1, wherein the first dictionary includes at least one of a diagnosis name dictionary containing names of diagnoses indicating lesions as the record information, a treatment name dictionary containing names of treatments indicating treatments involving an endoscope as the record information, or a treatment tool name dictionary containing names of treatment tools indicating endoscope treatment tools as the record information.

4. The information processing device according to claim 1, wherein the identifying characters include at least one of numerals, single letters of the alphabet, or abbreviations or common names indicating the record information.

5. The information processing device according to claim 1, wherein
the first dictionary is formed from a second dictionary in which identification information indicating the record information and the record information are registered in association with each other and a third dictionary in which the identifying characters and the identification information are registered in association with each other, and
the processor:
acquires the identification information associated with the identifying characters from the third dictionary on a basis of the recognized identifying characters; and
acquires the record information associated with the identification information from the second dictionary on a basis of the acquired identification information.

6. The information processing device according to claim 5, further comprising:
a graphical user interface (GUI), wherein
the processor newly creates the third dictionary or edits registered content of the third dictionary by operation input from the GUI.

7. The information processing device according to claim 1, further comprising:
a graphical user interface (GUI), wherein
the processor sets the first dictionary to enabled or disabled by operation input from the GUI.

8. The information processing device according to claim 1, wherein the processor:
acquires an endoscopic image during the endoscopy; and
enables the first dictionary when a specific type of photographic subject is detected from the endoscopic image.

9. The information processing device according to claim 1, wherein the processor:
acquires an endoscopic image during the endoscopy;
detects a type of lesion from the endoscopic image; and
sets the first dictionary to enabled or disabled according to the detected type of lesion.

10. The information processing device according to claim 1, further comprising:
a communication unit that communicates with a server that provides a speech recognition engine, wherein
the processor:
downloads or updates the speech recognition engine from the server through the communication unit; and
recognizes speech uttered by the user by using the downloaded or updated speech recognition engine.

11. The information processing device according to claim 1, wherein
the first dictionary includes a diagnosis name dictionary containing a plurality of names of diagnoses indicating lesions and a treatment tool name dictionary containing a plurality of names of treatment tools indicating endoscope treatment tools, and
the processor:
acquires an endoscopic image during the endoscopy;
recognizes at least one of a lesion or a treatment tool used in a treatment involving an endoscope, on a basis of the endoscopic image;
selects the diagnosis name dictionary or the treatment tool name dictionary on a basis of a result of recognizing the lesion or the treatment tool; and
acquires the record information corresponding to the identifying characters from the selected dictionary on a basis of the recognized identifying characters.

12. The information processing device according to claim 1, wherein the processor, upon recognizing speech expressing a wake word during the endoscopy, recognizes speech expressing the identifying characters uttered thereafter.

13. The information processing device according to claim 12, wherein
the first dictionary includes at least one of a diagnosis name dictionary containing a plurality of names of diagnoses indicating lesions, a treatment name dictionary containing a plurality of names of treatments indicating treatments involving an endoscope, or a treatment tool name dictionary containing a plurality of names of treatment tools indicating endoscope treatment tools,
the wake word is a word specifying at least one dictionary from among the diagnosis name dictionary, the treatment name dictionary, and the treatment tool name dictionary, and
the processor acquires the record information corresponding to the identifying characters from the dictionary specified by the wake word, on a basis of the recognized identifying characters.

14. The information processing device according to claim 1, further comprising:
a second display device independent from a first display device on which an endoscopic image is displayed during the endoscopy, wherein
the processor displays the first dictionary on the second display device during the endoscopy.

15. The information processing device according to claim 14, wherein the processor displays on the second display device at least one of a result of recognizing the speech uttered by the user or the acquired record information.

16. The information processing device according to claim 1, further comprising a masking sound generating device that generates masking sound that inhibits an ability of a patient to hear the speech uttered by the user during the endoscopy.

17. A tablet terminal comprising the information processing device according to claim 1.

18. An operating method for an information processing device comprising a processor and a first dictionary in which record information to be recorded in relation to endoscopy is registered,
the first dictionary being configured such that identifying characters that differ from the record information and the record information are associated directly or indirectly,
the operating method comprising:
recognizing, by the processor, speech which is uttered by a user during endoscopy and which expresses the identifying characters; and
acquiring, by the processor, the record information corresponding to the identifying characters from the first dictionary on a basis of the recognized identifying characters.

19. A non-transitory and computer-readable tangible recording medium in which a program for causing a processor provided to an information processing device to execute the operating method for an information processing device according to claim 18 is recorded.
